(19) United States
(12) Patent Application Publication
(10) Pub. No.: US 2010/0292580 A1
(43) Pub. Date: Nov. 18, 2010

(54) SEPTIC BARRIERS AND OTHER AIDS FOR AUTOFLUORESCENCE VISUALIZATION AND OTHER OPTICAL INTERROGATION

(75) Inventors: Terence J. Gilhuly, Vancouver (CA); Scott Benjamin, Hancock, NY (US); David Morgan, Richmond (CA); Pierre Lane, Vancouver (CA); Peter Whitehead, West Vancouver (CA); Ricardo Romero, Richmond (CA)

Correspondence Address: GRAYBEAL JACKSON LLP, 400 - 108TH AVENUE NE, SUITE 700, BELLEVUE, WA, 98004, US

(73) Assignee: LED Medical Diagnostics, Inc., White Rock, BC (CA)

(21) Appl. No.: 12/447,194

(22) PCT Filed: Oct. 25, 2007
(86) PCT No.: PCT/US07/22654

§ 371 Date: Jul. 30, 2010

Related U.S. Application Data
(60) Provisional application No. 60/854,579, filed on Oct. 25, 2006.

(51) Int. Cl. A61B 19/08; A61B 19/10; A61B 1/32; A61B 6/00

(52) U.S. Cl. 600/476; 128/853; 128/852; 600/201; 600/242; 600/202

(57) ABSTRACT

Septic barriers interposed between a target tissue such as an oral or vaginal cavity, exposed epidermis, or wound or surgical site, and a user and his/her optical interrogation instruments to reduce cross-infection and/or contamination. In certain embodiments, the septic barrier is substantially transparent for viewing purposes and is typically effectively non-fluorescent, particularly when used for investigating autofluorescence or other fluorescence emanating from a target, so as to have little or no effect on the measurements and observations being made. The sepsis barrier can comprises a window through which the user sees the tissue, a frame holding the window, and an attachment structure, such as threads or bayonet attachment, configured to connect the barrier to the instrument. If desired, the system can also have an optional attachment mechanism for permanent or temporary connection of a further instrument to the frame, such as a tissue retractor configured to aid manipulation of the target tissue.

### CROSS-REFERENCE TO RELATED APPLICATIONS

The present application claims priority from U.S. provisional patent application No. Ser. No. 60/854,579, filed 25 Oct. 2006, which is incorporated herein by reference in their entirety and for all their teachings and disclosures.

The present application does not claim priority from but does also incorporate by reference in their entirety, U.S. Pat. No. 6,021,344; U.S. Pat. No. 6,110,106; published US patent application US-2005-0234526-A1; published US patent application US-2006-0241347; U.S. patent application Ser. No. 11/402,284, filed Apr. 10, 2006; and, U.S. patent application Ser. No. 11/786,462, filed Apr. 10, 2007.

### BACKGROUND

Detection of cancer, including precancerous and early cancerous cells, as well as other diseases, has always been a difficult and uncertain process. One of the approaches to identifying cancerous cells has been to measure the cells' autofluorescence signature, because cancerous cells have a distinct autofluorescence signature relative to healthy cells. Other diseases also have changes in their autofluorescent signature. However, the detection of autofluorescence in traditional dental and medical environments has been problematic because the autofluorescence signature itself is very, very small compared to the ambient light typically present in an examination room, operating room, etc.

The following are some references relating to the detection of cancer in the oral cavity and other locations. As with all other references cited herein, including in the Cross Reference to Related Applications, these references are incorporated herein in their entirety for all their teachings and for all purposes. Zheng, W., et al., Detection of squamous cell carcinomas and pre-cancerous lesions in the oral cavity by quantification of 5-aminolevulinic acid induced fluorescence endoscopic images, Lasers Surg. Med. 31:151-157, 2002; Utzinger U, et al., Optimal visual perception and detection of oral cavity neoplasia, Cancer 2003 Apr. 1; 97(7):1681-92; Muller, M G, et al., Spectroscopic detection and evaluation of morphologic and biochemical changes in early human oral carcinoma, Cancer 2003; 97:1681-92; Majumder, S., Nonlinear pattern recognition for laser-induced fluorescence diagnosis of cancer, Lasers Surg. Med. 33:48-56, 2003; Tsai, T., et al., In vivo autofluorescence spectroscopy of oral premalignant and malignant lesions: Distortion of fluorescence intensity by submucous fibrosis, Lasers Surg Med 2003; 32(1):17-24; Ebihara, A, Detection and diagnosis of oral cancer by light-induced fluorescence, Lasers Surg. Med. 32:17-24, 2003, PMID: 12516066; Zheng W, Detection of neoplasms in the oral cavity by digitized endoscopic imaging of 5-aminolevulinic acid-induced protoporphyrin IX fluorescence, Int J Oncol 2002 October, 21(4):763-8; PMID: 12239614; Wang C Y, Autofluorescence spectroscopy for in vivo diagnosis of DMBA-induced hamster buccal pouch pre-cancers and cancers, J Oral Pathol Med 2003 January; 32(1):18-24, PMID: 12558954; Onizawa K, Characterization of autofluorescence in oral squamous cell carcinoma, Oral Oncol 2003 February; 39(2):150-6, PMID: 12509968;

Accordingly, there has gone unmet a need to improve the ability of a doctor, dentist or other person to diagnose cancerous or precancerous cells or other disease sites in a target tissue in a typical medical or dental setting, including for example the visualization and measurement of fluorescence such as tissue autofluorescence in medical applications. For example, there is a need for an improved septic barrier interposed between the subject tissue and the user or the user's instruments to inhibit cross-infection and contamination. The present systems, devices, methods, etc., herein provides these and/or other advantages.

### SUMMARY

The present systems, devices, methods, etc., in one aspect provide septic barriers interposed between a target tissue such as an oral or vaginal cavity, exposed epidermis, or wound or surgical site, and a user and his/her instruments to reduce cross-infection and/or contamination. In certain embodiments, the septic barrier is substantially transparent for viewing purposes and is typically effectively non-fluorescent, particularly when used for investigating autofluorescence or other fluorescence emanating from a target, so as to have little or no effect on the measurements and observations being made. In one embodiment, the sepsis barrier comprises a window through which the user sees the tissue, a frame holding the window in place and/or mounted, and an attachment structure or mechanism, such as threads or bayonet attachment, configured to connect the barrier to the instrument or equipment to be kept septic. If desired, the system can also have an optional attachment mechanism for permanent or temporary connection of a further instrument to the frame, such as a tissue retractor configured to aid manipulation of the target tissue. Unless expressly stated otherwise or clear from the context, all embodiments, aspects, features, etc., can be mixed and matched, combined and permuted in any desired manner.

In one aspect, the present methods, devices, etc., herein provide sepsis barriers configured to be disposed between a patient and a medical optical interrogation device, the barrier can comprise a substantially optically transparent and substantially non-fluorescent, thin plastic film window configured to be attached to the optical interrogation device. The window of the sepsis barrier can be angled relative to the viewing axis of the optical interrogation device to reduce reflection, can be removably or permanently attached to the frame of the sepsis barrier. The window of the sepsis barrier can be unitary, one-piece construction with the sepsis barrier.

The window of the sepsis barrier further can comprise an anti-fog coating, can comprise an attachment mechanism for a retractor configured to extend distally from the barrier and to manipulate the target tissue, which retractor can comprise a distal portion and proximal portion. The distal portion of the retractor can be shaped for the favorable retraction of cheek tissue to aid visualization of tissues, for example on the buccal side of the teeth. The sepsis barrier and the retractor can be reversibly combined, with the sepsis barrier providing a frame for the temporary or permanent anchoring of the retractor, and the combined device can be removably attachable to the distal end of the optical interrogation device. The interface of a combined anti-sepsis-retractor device with the optical interrogation device can be such that attachment of the combined device to the optical interrogation device can be restricted to a set of predefined angles about the visual axis. The retractor for retraction of tissue further can comprise markings for measurement of feature size, which can comprise at least one of linearly arranged holes or pits on a distal portion of the retractor. The holes and/or pits can be at least partially filled with a material different from the material of the mechanism for retraction of tissue.

The sepsis barrier can be configured to be used only one time then disposed. The sepsis barrier can comprise at least one structural element that can be altered or destroyed during or after the one-time use.

In another aspect, the devices, methods, etc., herein provide medical optical interrogation devices that can comprise a directional mirror suitable for use for combination of the illumination and viewing paths in the optical interrogation scopes herein, wherein two or more of the filters used for removing selected bandwidths from the emitted light can be combined on a single substrate. The directional mirror can be a dichroic filter for reflecting substantially blue light and the filter for removing selected bandwidths can be a yellow band-stop filter. The combined mirror and one or more filters can be constructed by depositing interference gratings onto a substrate. The combined mirror and one or more filters can be also constructed by depositing one or more interference gratings for the filter aspect onto a dichroic filter substrate, or by depositing one or more interference gratings for the dichroic filter aspect onto a pre-constructed filter substrate. The combined mirror and one or more filters can also be constructed by depositing one or more interference gratings to opposite sides of the same substrate.

In still a further aspect, the devices, methods, etc., herein provide sepsis barriers configured to be disposed between a patient and a medical optical interrogation device, the barrier can comprise a window angled relative to the optical viewing axis of the optical interrogation device to reduce reflection. The sepsis barrier further can comprise angled inner walls relative to the optical viewing axis of the optical interrogation device to reduce reflection. The size of the angle can be inverse to the length of the optical axis, and the size of the angle can be less than 20 degrees.

These and other aspects, features and embodiments are set forth within this application, including the following Detailed Description and attached drawings.

### BRIEF DESCRIPTION OF THE DRAWINGS

FIG. 1A depicts a front elevational perspective view of a sepsis barrier including a transparent window.

FIG. 1B depicts a rear elevational perspective view of a sepsis barrier including a transparent window.

FIG. 2 depicts a side cutaway view of a window of the sepsis barrier slanted relative to the viewing device to which the sepsis barrier is attached.

FIG. 3 depicts a side plan cutaway view of a sepsis barrier comprising sloping internal walls.

FIG. 4 depicts a perspective view of a tissue retractor for oral application with markings made by removing material in the retractor

FIG. 5 depicts a partial side elevational view of a viewing instrument and a threaded connection mechanism that mates with accepting threads on the sepsis barrier.

FIG. 6 depicts a perspective partial view of an exemplary sepsis barrier in position on an oral interrogation device for an oral application.

FIG. 7 depicts a side plan view of an exemplary sepsis barrier in position on an oral interrogation device for an oral application.

### DETAILED DESCRIPTION

The present systems, devices and methods provides approaches to enhance the operability and/or performance of medical optical interrogation devices such as endoscopes such as the VELscope® brand hand-held optical interrogation scope, certain aspects of which are discussed in US patent application US-2005-0234526-A1. The systems, devices and methods, etc., include improved sepsis barriers, improved optical element configurations and improved windows for sepsis barriers.

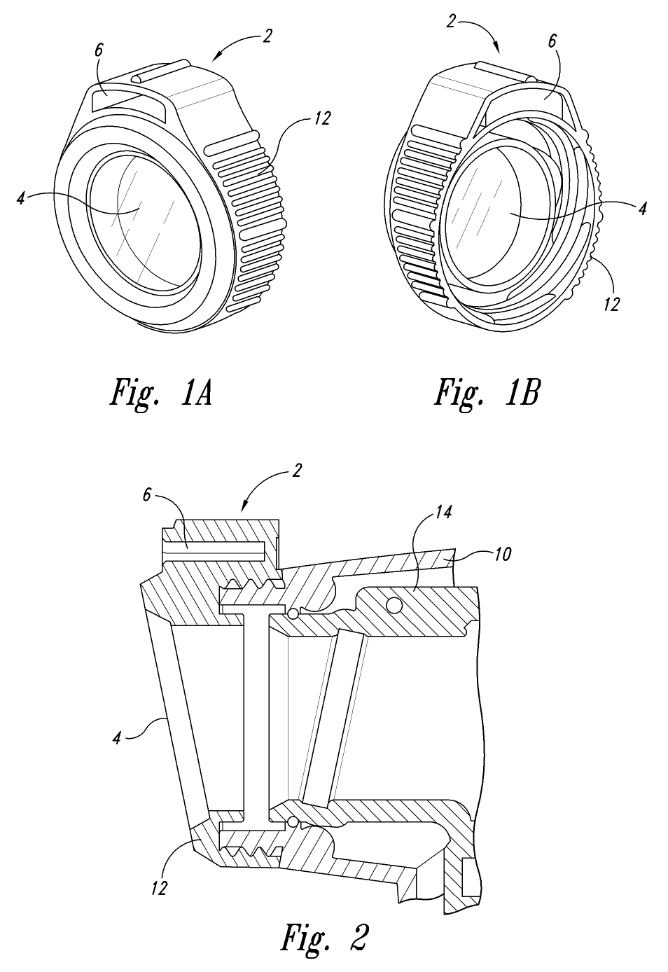

Turning to the Figures, FIGS. 1A and 1B depict front and rear perspective views of a sepsis barrier 2 including a transparent window 4. The front view, left, shows a window 4 and an optional mounting slot 6 for a tissue retractor, and the rear view, right shows an attachment system 8 comprising threads in the embodiment shown, for removably mounting the sepsis barrier 2 to a viewing instrument, i.e., medical optical interrogation device 10. Permanent mountings can be achieved as well, as well as other attachment systems 8 such as bayonet-type systems (similar to the configurations used to attach certain lenses to photographic cameras), form-fit systems, detents, etc.

The frame 12 of the barrier can be made of a substantially rigid material to allow removable attachment to the device for which it is being used as a sepsis barrier 2, and to withstand the forces imposed by manipulation of the target tissue when a tissue retractor is attached.

In an exemplary autofluorescence measurement situation (as well as a test or observation situation or other relevant situation), light at wavelengths causing fluorescent excitation of the target are transmitted through a light guide and/or directly to the target.

The target absorbs some of the light then releases it at longer, lower energy wavelengths as autofluorescence, and some of the light is reflected from the surface (there can also be transmission through and absorption by the target). If desired, additional fluorescent agents can be added to the tissue to provide additional fluorophores, for example for diagnostic or treatment purposes.

In one embodiment, as shown in FIG. 2, the window(s) 4 of the sepsis barrier 2 can be slanted relative to the medical optical interrogation device 10 to which the sepsis barrier 2 is attached and/or the tissue. This can decrease light reflected back into the viewing device 10 from the window 4 itself, for example excitation light being reflected from the inner surface of the window 4 back through the medical optical interrogation device 10, as well as reducing transmission of light reflected back from the target tissue then back through the medical optical interrogation device 10 (and thus back to the user, camera, etc.). This reduction in reflection can in turn decrease a signal that may otherwise occur due to the reflected light.

The angle(s) of the windows can if desired be matched trigonometrically to the length of the optical axis in the scope. This can inhibit reflections from reaching the user's eye by absorbing the reflected light into the body of the scope. In such embodiments, it is advantageous for the interior surface(s) of the scope to be coated with or otherwise comprise a light absorbing material 14.

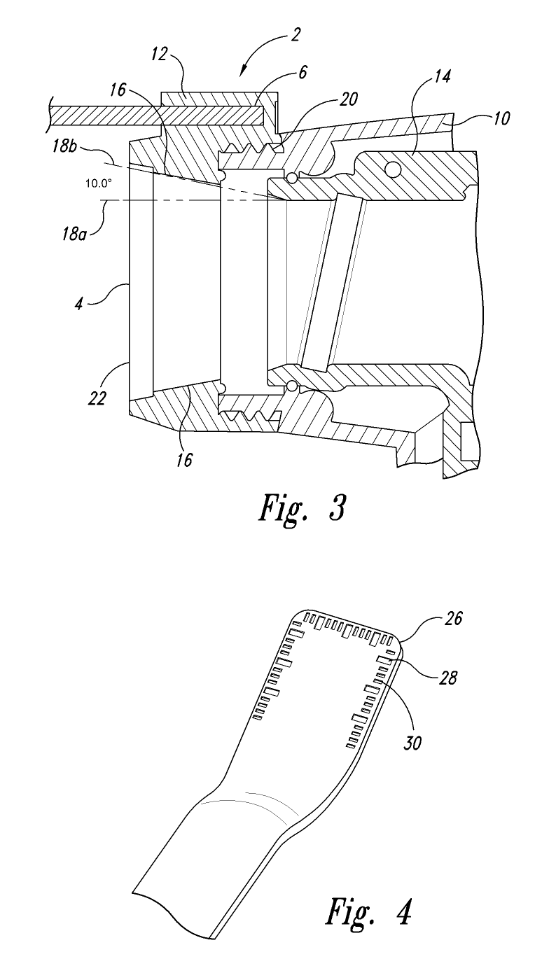

In certain embodiments, the light from the light sources and scopes, which can be collimated or non-collimated, is emitted as a cone. Thus, a sepsis barrier 2 with a straight tubular structure risks having some of its material receive and respond to excitation light, thereby producing extraneous fluorescence that may influence the measurement and observation. FIG. 3 depicts a side plan cutaway view of a sepsis barrier 2 comprising sloping internal walls 16. This changes the light pathway 18a, 18b into a conical shape and reduces the amount of light striking the inner walls 16 of the sepsis barrier 2, which also reduces extraneous fluorescence light. In FIG. 3 the sepsis barrier 2 has been given an exemplary wall angle of 10° to accommodate the output cone of light.

Turning to a further discussion of the window 4 in the sepsis barrier 2, in photonics and autofluorescence research and industry, typical windows have been a window of a non-fluorescing material such as BK-7 glass isolating the subject from the user. When the procedure or experiment is finished the window is either disposed of or cleaned, both of which can be costly. Cleaning also has the problems of guaranteeing sterility that is associated with re-use of medical devices. In some embodiments, the current devices, systems, etc., reduce such costs and inconvenience by providing disposable sepsis barriers such as those depicted in FIGS. 1 and 2 comprising thin plastic film sepsis barriers for use in autofluorescence detection and measurement. The use of plastic allows production of devices very cheaply, which permits single-use and then disposal. Disposability eliminates the problems of cross-contamination between patients and the costs associated with cleaning. In some embodiments, the sepsis barriers are specifically configured to be one-time use only disposable devices. In such embodiments, the devices can comprise one or more elements that are naturally altered or destroyed such as tab 20, which in the embodiment shown is crushed by the force of threading onto the optical interrogation device 10, so that a second, accidental use of the barrier is difficult or impossible, or at least likely to be noticed as a second use. This can advantageously help reduce the possibility of an anti-sepsis device herein being used first in a diseased patient and then inadvertently in a healthy patient with the unfortunate side effect of transmitting the disease to the healthy patient.

“Thin” in this context indicates the plastic film is thin enough that any autofluorescence generated by the plastic is so minimal that it does not significantly interfere with the interrogation of the tissue and the observation of the autofluorescence response. The use of such plastic has been discouraged in the past for autofluorescence interrogation of tissue, so the current methods, devices, etc., contradict and overcome this understanding to provide a suitable, inexpensive, advantageous product. In other words, plastics can fluoresce and can do so at a level that will reduce the signal to noise ratio from the sample being examined or otherwise interfere with the examination, depending on the plastic used, so the “thin” plastic film windows herein overcome this problem. This can reduce noise by reducing the amount of material in the excitation (and emission) pathway. The thickness can be a compromise between producing fluorescence of lower magnitude than that of the tissue being observed and the need for structural strength in window assembly. The window 4 can be made thin enough that it is a film, much like cellophane in form although various plastics (e.g., PVC or other desired materials) can be used.

In another embodiment, the window 4 is made of substantially low fluorescence plastic material such that the fluorescence produced by the plastic material is of lower magnitude than that of the tissue being observed. In some embodiments, the window 4 consists essentially of only a thin, very low fluorescence plastic film.

The window 4 can be coated with or otherwise comprise an anti-fogging material 22 to prevent the patient's breath or other humidity from fogging the window 4 and interfering with the measurement or observation. In other embodiments, for example where the window 4 consists essentially of only a thin, very low fluorescence plastic film, the window 4 specifically does not include an anti-fogging coating but anti-fogging effect is achieved due to the structure and composition of the material of the window 4 itself.

Several methods of attaching the window 4 to the frame 12 of the sepsis barrier 2 are possible, including: heat sealing, spin welding, ultrasonic welding, and glues. The window 4 can also be held to the sepsis barrier 2 by electrostatic charge as is done with various stickers and labels in the commercial electronics industry, or otherwise as desired.

FIG. 4 depicts a perspective view of a tissue retractor 26 for oral application with markings 28, 30 made by removing material in the retractor; the markings can also be made with ink or other coatings, including indelible inks, fluorescent inks, etc., or otherwise as desired. The tissue retractor 26 is a device useful to increase viewing at the target tissue. And can also be configured for other cavities or situations, such as vaginal, anal, wounds, and surgical sites.

In one embodiment, a cheek retractor 26 is disposed on the distal end of the scope. In patients' mouths there are areas that are difficult to access by viewing alone.

The cheek or tongue retractor enhances the ability to access such tissues, and gives the clinician a “third hand” —one to hold the scope, the second to hold the tongue and the “third” to retract the cheek. This can be particularly important if the clinician must work alone.

It can be important to be able to assess the size of lesions and other features in and on the target tissue. This can be done in some embodiments, such as shown in FIG. 4, by having the cheek retractor 26 (or other structure if no retractor 26 is present) further comprise markings of distance for assessment of size, such as a measurement scale. These markings can be implemented, for example, by overmolding materials of different fluorescent properties to that of the tissue retractor 26; using fluorescent paints/glues; by having the tissue retractor 26 comprise a hollow body with material located in differing quantities throughout the part; by doping the material in select locations with fluorophores; by etching the tissue retractor 26; by burning the tissue retractor 26 material if light; by bleaching the tissue retractor 26 (photobleaching or chemical) if dark; and/or by cutting holes through and/or cutting pits into the tissue retractor 26 and using the difference in material height and/or edges to provide visible contrast. The markings can also include shapes or other configurations in addition to or instead of distance markings, for example shapes or pictures of what particular lesions look like with identifying information next to the picture.

Turning to various approaches to attaching the retractor 26 to the optical interrogation device 10, the retractor 26 is typically attached to prevent slippage during use and to provide defined angles of application for location of features found during examination and documentation purposes. The optical interrogation device 10 can be a handheld oral scope as discussed in US patent application US-2005-0234526-A1. The attachment of the retractor 26 to the device can be, if desired, restricted to a small set of predefined angles of roll about the visual axis, for example using a set of detents, a rack and pawl, a rack and pinion, a button that activates or releases a biasing spring, a lever, or other device or system that moves the barrier or window 4 from one incremental position to another. The angle of roll about the visual axis of the cheek retractor 26 can be changed by ratcheting between the positions; or removal of the sepsis barrier 2, modification of orientation, and reapplication, or otherwise as desired.

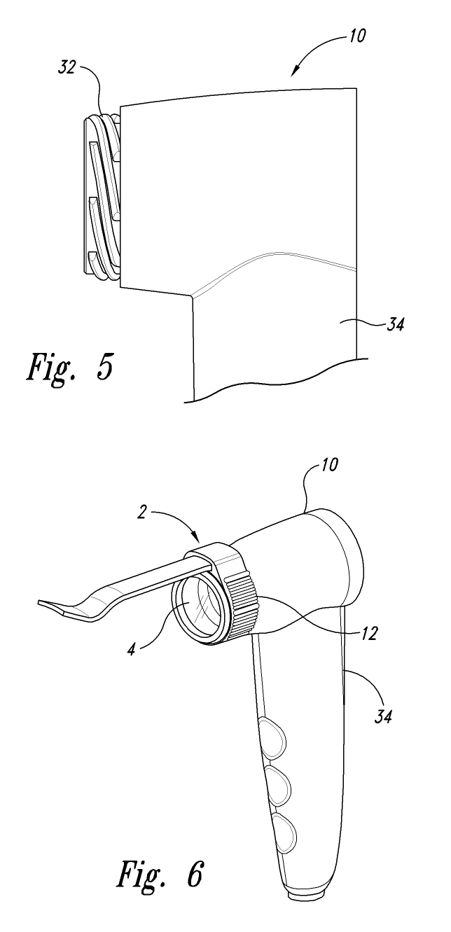

Various methods of attaching the sepsis barrier 2 to the device can be implemented, including: screw on, snap on, pinch cap gripping, automatic attachment of the sepsis barrier 2 upon insertion of the handheld device into a specialize holder. This last method can be similar to what is done with disposable vials for pipettes. FIG. 5 depicts a partial side elevational view of a medical optical interrogation device 10 having a threaded connection mechanism 32 that mates with accepting threads on the sepsis barrier 2 and a handle 34 to facilitate use of the hand-held version depicted.

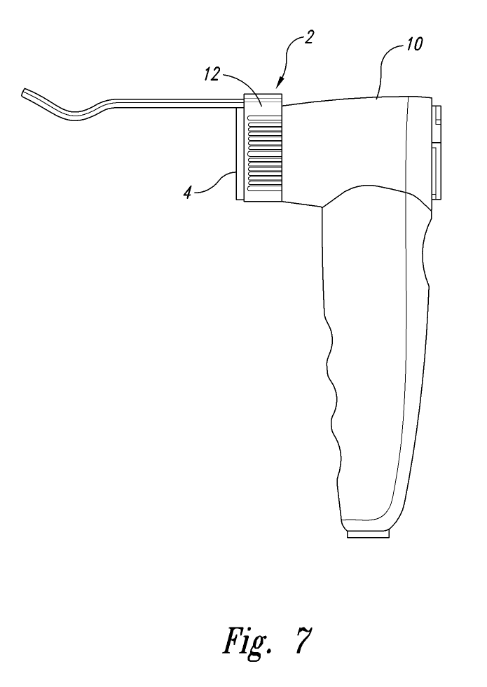

FIGS. 6 and 7 depict an exemplary sepsis barrier 2 in position on an oral optical interrogation device 10 for an oral application. A tissue retractor 26 is shown that would be used in retracting cheeks, the tongue, etc. The shape and size of this retractor has been developed specifically for cheeks and can be revised and shaped and sized differently for other tissues as desired, although the configuration depicted, with the curve at the distal end and the distal tip projecting above the level of the plane of the stem of the retractor 26, can also be used with other tissues if desired.

The systems, methods, devices, etc., herein can be used for providing barriers for assessment of cancerous or otherwise malignant tissues in the mouth, vagina, esophagus, lung; delineation of surgical margins for surgeries in which malignant tissues are to be removed; detection of bacteria, or otherwise as desired.

In one embodiment, the systems, methods, devices, etc., herein can comprise a removable single-use window 4, and the frame 12 holding the window 4 can be cleanable and have a longer (extended) lifetime. The window 4 can then be easily removable from the frame 12 and discarded. This allows maintenance of the sepsis barrier 2 with reduced cost as less material is disposed of per patient, and adds the benefit of reduced storage volume as the windows 4 can be made stackable and fit more closely together this way.

One method of implementation is to have the windows 4 as thin plastic disks such as a thin film that are inserted into a groove and held with a friction fit. Another is to have the windows as plastic disks that are press-fit behind tabs. Clips (spring clips, rings or other related devices) can be used to hold the disk against the frame 12. The disk can be simple and flat, or can have features such as grooves and ridges to increase the grip of the frame 12 and/or increase the convenience of application.

The windows 4 can also be made to screw attach to the frame 12. This can be as the round disk with a threaded outer circumference and pegs or holes to allow rotational attachment by hand or with a tool. As well, this can be in the shape of a bottle cap with either internal or external thread to allow grip of protruding or intrinsic features on the sepsis barrier frame 12.

The sepsis barrier 2 can be supplied in a stackable format similar to what is done for “Dixie™ cup” water cups. This stacking can reduce the total volume of the packaging by having the devices mate together as stacks and provided in a many-to-one package. The many-to-one package can allow the sepsis barriers 2, typically disposable, to be made available in an organized format such that the user can attach the sepsis barrier 2 using only one hand. A suitable dispensable format is such that the sepsis barriers 2 are stacked, one on top of the other, with the outermost sepsis barrier 2 arranged ready for attachment, for example by projection and detent, bayonet style or otherwise as desired.

Furthermore, methods of sepsis barrier 2-removal can be provided such that removal can also be done single-handedly. For example, a trigger connected to a spring, pushrod, or airtight bellows can be used to force the sepsis barrier 2 off when no longer required. This can be done over a disposal unit so the user would not have to contact the contaminated sepsis barrier 2.

In another aspect, the systems, methods, etc., herein comprise a combined illumination and viewing light path, wherein it can be helpful to remove unwanted light to each of illumination and viewing. In one embodiment, as discussed in US patent application US-2005-0234526-A1, there is a dichroic mirror to pass only blue (excitation) light from a light source to the tissue and prevent reflected blue light from reaching the user by being reflected back toward the light source. Longer-than-blue wavelengths (i.e., the measured fluorescence) are passed through the mirror to the user. To improve diagnosis, the fluorescent light was further filtered to remove yellow light and thereby increase the contrast between the green and red reflected light. This was done using a yellow band-stop filter (specific wavelengths were 590+/−30 nm).

To reduce cost of parts and to reduce the size of the instrument, the filter and mirror can be combined into a optical single piece. To make both the filter and mirror separately, interference gratings (patterned layers) are typically deposited onto a glass substrate. To make a single optical element comprising both the filter and the mirror, the desired layers to provide the filter functions can be applied to the already-built mirror once it has been built or instead the layers defining the dichroic function can be applied to the already-built filter. Alternatively, the filter and mirror could be added to opposite sides of the same substrate.

Generally speaking, filters of this construction are designed for angles of light incidence of zero degrees. Should this angle be changed by tilting the filter, the grating pattern must also be changed to maintain desired functionality. For example, if the dichroic mirror is angled 45 degrees (the number can differ depending on desired design) to complete its function of reflection, the filter combined with the dichroic mirror would have a corresponding modification of its design.

Another useful quality that can be obtained using interference gratings is attenuation. This is done to create neutral density (broad spectrum, typically covering the visible spectrum) filters. Thus, another option is to make combination filters with attenuation as well. This can be, for example, combined mirror and attenuators, combined narrowband filters and attenuators, and a combination of all three aspects mirror, filter and attenuation.

As noted above, to aid discrimination between healthy and unhealthy tissue, a bandstop filter can be employed. This filter reflects or intercepts light in the stop band and allows other wavelengths to pass through. For example, light emanating from the tissue can be reflected back towards the tissue and light from the external environment can be reflected back towards the external environment. In practice, this resulted in a reflection—generally of the User's eye—appearing in the field of view. Thus the user typically used the optical interrogation device 10 close to his or her own eye(s) to block out all the background light. This can be uncomfortable for the user should they have back problems and uncomfortable for the patient in a more social sense. To remedy this problem, in one embodiment of the devices, etc., herein, the last optic in the eyepiece closest to the user (i.e., the most proximal optical element) can be angled such that the reflection is reflected into the walls of the eyepiece or to some other unimportant location. Trigonometry can be used to determine suitable angle(s) of the optic element and the length of the eyepiece to fully capture the reflection. In another embodiment, an opaque eyecup can be provided, with or instead of the angling of the optical element, to reduce light impacting the eye of the user. In one embodiment to decrease the angle needed to reflect ceiling light into the walls of the eyepiece and thereby the length of the eyepiece, it was assumed that most external light would be coming from overhead lights, the optic was tilted downwards to preferentially reflect ceiling light. Incorporating the band-stop filter at 45 degrees as part of the dichroic mirror is a embodiment for suppressing the back reflection from the filter reaching the user's eye because it buries the unwanted light inside the device at roughly the midpoint of the horizontal optical axis thus inhibiting back reflections from the above mentioned arguments of trigonometry.

The scope of the present devices, systems and methods, etc., includes both means plus function and step plus function concepts. However, the claims are not to be interpreted as indicating a “means plus function” relationship unless the word “means” is specifically recited in a claim, and are to be interpreted as indicating a “means plus function” relationship where the word “means” is specifically recited in a claim. Similarly, the claims are not to be interpreted as indicating a “step plus function” relationship unless the word “step” is specifically recited in a claim, and are to be interpreted as indicating a “step plus function” relationship where the word “means” is specifically recited in a claim.

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims or other claim having adequate support in the discussion herein.

1. A sepsis barrier configured to be disposed between a patient and a medical optical interrogation device, the barrier comprising a substantially optically transparent and substantially non-fluorescent, thin plastic film window configured to be attached to the optical interrogation device.

2. The sepsis barrier of claim 1 wherein the window of the sepsis barrier is angled relative to the viewing axis of the optical interrogation device to reduce reflection.

3. The sepsis barrier of claim 1 or 2 wherein the window of the sepsis barrier is removably attached to the frame of the sepsis barrier.

4. The sepsis barrier of claim 1 or 2 wherein the window of the sepsis barrier is permanently attached to the frame of the sepsis barrier.

5. The sepsis barrier of claim 1 or 2 wherein the window of the sepsis barrier is unitary, one-piece construction with the sepsis barrier.

6. The sepsis barrier of claim 1 or 2 wherein a window of the sepsis barrier further comprises an anti-fog coating.

7. The sepsis barrier of claim 1 or 2 wherein the device further comprises an attachment mechanism for a retractor configured to extend distally from the barrier and to manipulate the target tissue.

8. The sepsis barrier of claim 1 or 2 further comprising a refractor for the retraction of tissue, clothing or other obstructions from a site of interest, the retractor comprising a distal portion and proximal portion.

9. The sepsis barrier of claim 8 wherein the distal portion of the retractor is shaped for the favorable retraction of cheek tissue to aid visualization of inner cheek tissues.

10. The sepsis barrier of claim 8 whereby the sepsis barrier of claim 8 and the retractor of claim 9 are reversibly combined, with the sepsis barrier providing a frame for the temporary or permanent anchoring of the retractor, and the combined device is removably attachable to the distal end of the optical interrogation device.

11. The sepsis barrier of claim 10 wherein the interface of the combined device to the optical interrogation device is such that attachment of the combined device to the optical interrogation device is restricted to a set of predefined angles about the visual axis.

12. The sepsis barrier of claim 8 wherein the retractor for refraction of tissue further comprises markings for measurement of feature size.

13. The sepsis barrier of claim 12 wherein the markings comprise at least one of linearly arranged holes or pits on a distal portion of the retractor.

14. The sepsis barrier mechanism of measurement of claim 13 wherein the holes and/or pits are at least partially filled with a material different from the material of the mechanism for refraction of tissue.

15. The sepsis barrier of claim 1 or 2 wherein the sepsis barrier is configured to be used only one time then disposed, wherein the sepsis barrier comprises at least one structural element that is altered or destroyed during or after the one-time use.

16. A medical optical interrogation device comprising a directional mirror suitable for use for combination of the illumination and viewing paths in the optical interrogation scopes herein, wherein two or more of the filters used for removing selected bandwidths from the emitted light are combined on a single substrate.

17. The medical optical interrogation device of claim 16 wherein the directional mirror is a dichroic filter for reflecting substantially blue light and the filter for removing selected bandwidths is a yellow band-stop filter.

18. The medical optical interrogation device of claim 16 or 17 wherein the combined mirror and one or more filters are constructed by depositing interference gratings onto a substrate.

19. The medical optical interrogation device of claim 16 or 17 wherein the combined mirror and one or more filters are constructed by depositing one or more interference gratings for the filter aspect onto a dichroic filter substrate.

20. The medical optical interrogation device of claim 16 or 17 wherein the combined mirror and one or more filters are constructed by depositing one or more interference gratings for the dichroic filter aspect onto a pre-constructed filter substrate.

21. The medical optical interrogation device of claim 16 or 17 wherein the combined mirror and one or more filters are constructed by depositing one or more interference gratings to opposite sides of the same substrate.

22. sepsis barrier configured to be disposed between a patient and a medical optical interrogation device, the barrier comprising a window angled relative to the optical viewing axis of the optical interrogation device to reduce reflection.

23. The sepsis barrier of claim 22 further comprising angled inner walls relative to the optical viewing axis of the optical interrogation device to reduce reflection.

24. The sepsis barrier of claim 22 or 23 wherein the size of the angle is inverse to the length of the optical axis.

25. The sepsis barrier of claim 22 or 23 wherein the size of the angle is less than 20 degrees.
